(19) United States
(12) Patent Application Publication
(10) Pub. No.: US 2013/0072578 A1
(43) Pub. Date: Mar. 21, 2013

(54) ORODISPERSIBLE TABLETS OF ERYTHRITOL AND ISOMALT

(71) Applicants: Catherine Patricia L. Boghmans, Beigem (BE); Liesbeth Maria Fernande Meeus, Everberg (BE)

(72) Inventors: Catherine Patricia L. Boghmans, Beigem (BE); Liesbeth Maria Fernande Meeus, Everberg (BE)

(21) Appl. No.: 13/701,051

(22) PCT Filed: May 17, 2011
(86) PCT No.: PCT/EP11/02432

§ 371 Date: Nov. 30, 2012

(30) Foreign Application Priority Data
Jun. 1, 2010 (EP) 10005677.9

(51) Int. Cl. A61K 47/26

(52) U.S. Cl. CPC A61K 47/26
USPC 514/777

(57) ABSTRACT

Erythritol is granulated together with at least 10% w/w isomalt. Prior and/or after granulation a disintegrant is added and orodispersible tablets are prepared. The tablet has a disintegration time of less than 100 seconds, less than 90 seconds, preferably less than 80 seconds, more preferably less than 60 seconds and said disintegration time was determined according to the European Pharmacopoeia VI, Test method 2.9.1 by using a pharmaceutical disintegration tester model ZT 73 whereby 6 tablets having a surface of 1 square centimeter and a weight of 350 mg, at a compression force of 20 kN, were analyzed and mean values were calculated. The process for preparing the orodispersible tablet, its use, and the intermediate granulate are described as well.

### TECHNICAL FIELD

The present invention relates to the preparation of an orodispersible tablet of erythritol, isomalt and a disintegrant.

### BACKGROUND OF THE INVENTION

Tablets and capsules have drawbacks in that water is needed when they are taken and they are not well-accepted by aged people, infants and those having difficulty in swallowing. It has been reported that Dysphagia (difficulty in swallowing) is common among all age groups and more specifically with pediatric, geriatric population along with institutionalized patients and patients with nausea, vomiting and motion sickness complications. During the last decade there is a need for an upcoming generation of pharmaceutical products and medicaments which can be taken anywhere. A suitable type of formulations are existing under the form of orodispersible form or are rapid dissolved and have the characteristics of dissolving, or melting or disintegrating in the oral cavity in only a few seconds in absence of water. These formulations are quickly disintegratable or soluble when put in the oral cavity, and thus are suitable for the aged people, infants and those having difficulty in swallowing.

WO 2010/001063 describes orodispersible mannitol under the form of a co-agglomerate of mannitol and granular starch.

WO 2010/025796 describes chewable tablets comprising erythritol having a specific surface area greater than 0.25 m²/g and a binder selected from the group consisting of pregelatinised starch, microcrystalline cellulose, carboxymethyl cellulose, maltose, sorbitol, maltitol, xylitol, isomalt, and mixtures thereof. The hardness and friability are highly important properties of a chewable tablet.

WO 2010/054845 describes calcium carbonate tablets comprising at least 50% calcium carbonate. It is shown in examples where isomalt and sorbitol have been chosen as binding sugar alcohols, the amount of sorbitol turns out to be critical resulting in unsatisfactory dissolution profiles.

EP 0 922 464 relates to a process for preparing quickly disintegradable compression-molded materials based upon erythritol. A tablet is obtained by compression molding. The thus obtained quickly disintegradable compression molded material is endowed with excellent disintegration and dissolution properties when put in the oral cavity or water.

There is a further interest for using erythritol and isomalt in orodispersible tablets.

### SUMMARY OF THE INVENTION

The current invention relates to an orodispersible tablet comprising a disintegrant, erythritol and at least 10% w/w isomalt, preferably at least 15% w/w, more preferably at least 20% w/w and these tablets have a disintegration time of less than 100 seconds, less than 90 seconds, preferably less than 80 seconds, more preferably less than 60 seconds, for tablets prepared with compression force of 20 kN with a surface of 1 square centimeter, and a weight of 350 mg.

It further describes a process for preparing the orodispersible tablet according to current invention.

It relates to tablets for use as medicament and the use of the orodispersible tablet in feed, cosmetic applications, personal care applications, detergent applications, nutritional supplements and agro-applications.

Finally it relates to a granulate of disintegrant, erythritol and from 10% w/w to 50% w/w isomalt.

### DETAILED DESCRIPTION OF THE INVENTION

The current invention relates to an orodispersible tablet comprising a disintegrant, erythritol and at least 10% w/w isomalt, preferably at least 15% w/w, more preferably at least 20% w/w and most preferably less than 50% w/w isomalt, and these tablets have a disintegration time of less than 100 seconds, less than 90 seconds, preferably less than 80 seconds, more preferably less than 60 seconds for tablets prepared with compression force of 20 kN with a surface of 1 square centimeter, and a weight of 350 mg.

The disintegration time was determined according to the European Pharmacopoeia VI, Test method 2.9.1 by using a pharmaceutical disintegration tester model ZT 73 whereby 6 tablets prepared at the same compression force were analyzed and mean values were calculated. Preferably the isomalt is present in an amount of less than 50% w/w.

Orally disintegrating tablets (=orodispersible tablets) are solid dosage forms that undergo a disaggregation in the mouth in contact with the saliva, usually in a matter of seconds, forming a suspension which is easy to swallow, and this without the need to take it with water or without chewing. Alternative definitions of orodispersible tablets are quickly disintegrating tablets, quickly dispersible tablets, mouth dissolving tablets, fast disintegrating tablets, fast dissolving tablets, fast melting tablets (rapimelts) or rapid dissolving tablets. Therefore, orodispersible tablets are a special type of tablets which are meant to disintegrate quickly and as such have a disintegration time of less than 100 seconds for tablets prepared with compression force of 20 kN and where the tablets have a surface of 1 square centimeter, and a weight of 350 mg. Longer disintegration times are not suitable for orodispersible tablets. Tablets which have a much longer disintegration time, such as above 150 seconds, while having the same dimensions and prepared under similar conditions of compression force (of 20 kN), are not suitable as orodispersible tablets.

The term “tablet”, as used herein, includes tablets in any form, shape and of any physical, chemical or sensory property, and tablets for orodispersible administration. The orodispersible tablet according to the present invention is a tablet that undergoes rapid disaggregation and releases the active ingredient, flavor, aroma or the like, in the mouth before swallowing. An orodispersible tablet dosage form can be a pill, tablet, gum and more recently orodispersible squares.

A super-disintegrant is also called a disintegrant and for ease of understanding the present invention is using the terminology of disintegrant for disintegrant per se and so-called super-disintegrants as well.

The purpose of a disintegrant is to facilitate the breakup of a tablet after administration. Disintegration efficiency is based on the force-equivalent concept (the combined measurement of swelling force development and amount of water absorption). Force equivalence expresses the capability of a disintegrant to transform absorbed water into swelling (or disintegrating) force. A disintegrant must quickly wick saliva into the tablet to generate the volume expansion and hydrostatic pressure necessary to provide rapid disintegration in the mouth.

Suitable examples of disintegrants (super-disintegrants) are calcium alginate, sodium alginate, calcium carboxymethyl cellulose, sodium carboxymethylcellulose, microcrystalline cellulose, methylcellulose, hydroxypropylcellulose, sodium croscarmellose (internally cross-linked sodium carboxymethyl cellulose), chitosan, colloidal silicon dioxide, povidone (polyvinylpyrrolidone), crospovidone, guar gum, magnesiumaluminiumsilicate, sodium starch glycolate, starch, mixture of two or more thereof, and the like.

Erythritol is well-known and is a tetriitol which is obtainable via microbial processes or fermentation, chemical processes, preferably other than just hydrogenation of carbohydrates. Most preferably fermentation is used for the production of erythritol. Any grade of erythritol is suitable and without any limitation, a suitable source of erythritol is a micronized erythritol prepared as described in WO2009016133, or a fine grade of erythritol, or preferably turbomilled erythritol and the like. Mixtures of different grades can be applied as well.

Isomalt is understood to refer to an almost equimolar mixture of 6-glucopyranosyl-sorbitol (6-GPS) and 1-glucopyranosyl-mannitol (1-GPM), and the weight percentage can vary between 43% to 57% of 6-GPS to 57% to 43% of 1-GPM. Any other ratio of both components is falling under the definition of the mixture containing 6-glucopyranosyl-sorbitol, and 1-glucopyranosyl-mannitol. These mixtures can be enriched in one of the component, be it 1-GPM or 6-GPS or another isomer, 1-glycopyranosyl-sorbitol (1-GPS) may be present as well. The mixtures containing 6-glucopyranosyl-sorbitol, and/or 1-glucopyranosyl-mannitol, as well as the isomalt may further comprise minor amounts of other substances such mannitol, sorbitol, hydrogenated or non-hydrogenated oligosaccharides as well as optionally glucose, fructose and/or sucrose, trehalulose, isomaltulose or isomaltose. Preferably isomalt containing an almost equimolar mixture of 6-glucopyranosyl-sorbitol (6-GPS) and 1-glucopyranosyl-mannitol (1-GPM) is used. Isomalt is present in an amount of at least 10% w/w, preferably at least 15% w/w, more preferably at least 20% w/w and preferably in an amount less than 50% w/w.

Furthermore, the orodispersible tablet is comprising the disintegrant in an amount of 0.5 to 20% w/w, preferably from 1 to 15% w/w, more preferably from 2 to 10% w/w. The actual content of the disintegrant depends upon the specific type used and also upon the point of addition in the process for preparing the orodispersible tablet of the current invention. For example, sodium croscarmellose is used in quantities of 0.5 to 5% w/w, whereas sodium starch glycolate is used in amounts of 1 to 20% w/w, calcium carboxymethyl cellulose is usually applied in a quantity of 1-15% w/w, sodium alginate in an amount of 2.5 to 10% w/w and microcrystalline cellulose in an amount of 5 to 15% w/w.

The tablet itself is further characterized in that it has specific properties in respect of tensile strength, moisture uptake, tablet porosity, wetting time, disintegration time and the like. Preferably these tablets have a surface of at least 1 cm² and a weight of 350 mg.

The tensile strength of these tablets can be expressed in function of compression force. A tensile strength at 15 kN of at least 2.2 N/mm², preferably, at least 2.4 N/mm², more preferably at least 2.5 N/mm², most preferably at least 2.7 N/mm² is obtainable, wherein said tensile strength (Ts), expressed as N/mm², is calculated as follows:

Ts=2H/πTD,

wherein H is the hardness, T the thickness and D the diameter of the tablet and wherein said hardness was determined according to the European Pharmacopoeia VI Test method 2.9.8 by using a pharmaceutical hardness tester model Multicheck V.

The tablets from the current invention, including a disintegrant have a lower tensile strength than the corresponding tablets (same polyol composition) without disintegrant. Usually a lower disintegration time corresponds to a lower tensile strength. As a tablet is less compacted, it is easier for the fluid to get into the tablet and induces disintegration of the tablet, and as such a good orodispersible tablet is obtained.

Whereas usually tablets may be characterized by their friability (=the ability of the compressed tablet to avoid fracture and breaking apart during transport) this parameter is less suitable for the evaluation of orodispersible tablets. The European Phramacopoeia VI has not yet included a limit for the friability of orodispersible tablets. The tablets of the current invention might easily have friability values (measured according to European Pharmacopoeia VI Test method 2.97) of at least 10%, even higher than 15%.

The current invention further relates to a process for preparing the orodispersible tablet of the current invention and it is characterized by a granulation step for preparing a granulate and followed by tabletting of the granulate.

Granulation methods can be divided in two basic types, namely wet methods, which use a liquid in the process, and dry methods in which no liquid is used. Wet granulation is most often used and involves different steps, including: agglomerating (granulating) of dry primary powder particles of active ingredients and excipients in the presence of a granulating fluid upon agitation using low-shear or high-shear mixers or fluidized beds, wet sieving (wet screening) to remove larger lumps, drying the granulated product, and milling or sieving (screening) the dried granulated product to achieve a granulated product having the desired granule size distribution. The obtained granulated product may subsequently be tabletted.

Isomalt is acting as a binder and can be added in dry or liquid form. The preferred binder is isomalt containing an almost equimolar mixture of 6-glucopyranosyl-sorbitol (6-GPS) and 1-glucopyranosyl-mannitol (1-GPM). Liquid isomalt is further containing 1-glycopyranosyl-sorbitol (1-GPS) in quantities of at least 2% based on dry matter.

The process is further characterized in that the disintegrant is added prior and/or after the granulation step.

By adding the disintegrant prior to the granulation step, quantities of disintegrant and addition are adapted such that the granulate is not yet disintegrating during preparation. Alternatively, the disintegrant is added after the granulation step. The quantities of the disintegrant are less affected by the process conditions and it may have a different effect on the tablet properties. Finally the disintegrant can be added prior and after granulation step.

The process is comprising the following steps:
a) taking erythritol, isomalt in dry or liquid form,
b) optionally adding water,
c) optionally disintegrant
d) granulating,
e) optionally wet sieving of granulated product,
f) drying the granulated product,
g) optionally sieving of the granulated product
h) blending with a lubricant, and optionally disintegrant
i) tabletting at compressing forces from 5 to 20 kN.

The fact that in step c) or h) the disintegrant is added optionally is referring back to the options to add the disintegrant prior and/or after the granulation step.

The binder, isomalt can be added in dry or liquid form. When adding isomalt in dry form, water is further added. Based upon the total dry matter of erythritol and isomalt, water is added in quantities of from 2% to 10%, preferably from 3% to 8%, most preferably in quantities at about 5% to 6%.

Depending upon the volume mean diameter and the moisture content of the blend, the granulate is sieved and/or dried.

The granulate formed in step d) of the current process is optionally pressed through a sieve of a predetermined size. Preferably a screening machine is applied for this sieving. At the same time or thereafter the product is dried.

Any drier type can be applied for drying of the granules, but preferably a fluid bed is applied for this purpose. The sufficiently dry product is granulated in a typical granulator.

The current invention further describes the granulate of disintegrant, erythritol and from 10% w/w to 50% w/w isomalt, and preferably the disintegrant is present in an amount of 0.5 to 5% w/w, preferably 1 to 2% w/w.

The granulate can be used in feed, pharma applications, cosmetics, detergents, fertilizer, agrochemical products and nutritional supplements. In fact, without being limiting, the compressible composition of the current invention can be used in nutritional supplements, animal feed, animal medicine, with bath agent, in agrochemical products, with fertilizer, with plant granules, with plant seeds or seed grains, and any other product being it ingested by humans and/or animals or any other product which can benefit from the orodispersible properties of the granulate of the current invention. The granulate of the current invention can be used as carrier for additives based on enzymes or microorganisms, detergent tablets, vitamins, flavors, perfumes, acids, sweeteners or various active ingredients with medicinal or non-medicinal applications. Eventually mixtures of additives can be applied.

The granulated product obtained in step d) of the current process is further blended with a suitable lubricant and optionally disintegrant and tabletted in a tabletting machine. Depending upon the addition point of the disintegrant, the granulate product is either containing disintegrant and no further disintegrant is added before tabletting, or the granulate is not yet containing disintegrant and disintegrant is added before the tabletting. Finally the granulate may contain disintegrant and further disintegrant is added before tabletting.

As a lubricant agent in tablet formation, magnesium stearate, calcium stearate, stearic acid, sucrose fatty acid esters, and/or talc and the like can be added according to needs. Furthermore surface active agents such as sodium lauryl sulfate, propylene glycol, sodium dodecanesulfonate, sodium oleate sulfonate, and sodium laurate mixed with stearates and talc, sodium stearyl fumarate, sucrose fatty acid esters, and the like can be added according to needs. Preferably magnesium stearate is used.

Finally it relates to a tablet for use as a medicament and the use of tablet in feed, cosmetic applications, personal care applications, detergent applications, nutritional supplements and agro-applications.

If tablets are prepared for pharmaceutical applications an active ingredient such as a drug is added and fillers, and/or lubricating agents are added if needed.

The tablets prepared according to the current invention are based upon a granulate of disintegrant, from 50% w/w to 90% w/w erythritol and from 10% w/w to 50% w/w isomalt, and preferably the disintegrant is present in an amount of 0.5 to 5% w/w, preferably 1 to 2% w/w.

The invention will hereunder be illustrated in the form of a series of non-limiting examples.

### EXAMPLES

### Methods for Evaluating Granule and Tablet Properties

The granules were characterized by their volume mean diameter (size distribution). The following measurement method was employed.

Size Distribution.

Size distribution was determined according to the European Pharmacopoeia VI Test method 2.9.31 using a laser light particle sizer, type Helos KF—Rodos T4.1, of Sympatec GmbH (Germany). The particle size was analysed by laser light diffraction.

The tablets were characterized by their hardness and disintegration time. For each compression force, 10 tablets for hardness and 6 tablets for disintegration time were analyzed and mean values were calculated. The following measuring methods were employed.

Hardness.

Hardness, i.e. the diametral crushing strength, was determined according to the European Pharmacopoeia VI Test method 2.9.8 Resistance to crushing of tablets by using a conventional pharmaceutical hardness tester (hardness tester model Multicheck V, available from Erweka GmbH (Germany)). In order to compare values across different size tablets, the breaking strength was normalized for the area of the break. The normalized value, expressed as N/mm², is herein referred to as tensile strength (Ts) and calculated as follows:

Ts=2H/πTD,

wherein H is the hardness, T the thickness and D the diameter of the tablet. For each compression force, 10 tablets were analyzed on hardness (H), thickness (T) and diameter (D).

Disintegration Time.

The disintegration time, i.e. the time needed to break up the tablet in a liquid medium, was determined according to the European Pharmacopoeia VI, Test method 2.9.1 Disintegration of Tablets and Capsules by using a conventional pharmaceutical disintegration tester (disintegration tester model ZT 73, available from Erweka GmbH (Germany)).

### Example 1

Coarse erythritol product (Cargill Zerose™ 16957) was milled in a Bauermeister turbo mill UTL at a 1 mm sieve and powder with a volume mean diameter of 25 μm was obtained. The volume mean diameter was determined with laser diffraction.

400 g of the milled erythritol powder was dry blended in a high Shear Mixer (Pro-C-ept-Mi-Pro, Chopper: 3000 rpm and Impeller: 1200 rpm) with 100 g isomalt (Cargill C*IsoMaltidex™) for 10 seconds.

30 ml of water was added in droplets at 5 ml/min. After the addition of the liquid, the mixing of the blend was continued for 60 seconds.

The granulated powder was manually wet screened over a 2 mm sieve.

The wet sieved granules were dried in the fluid bed (Aeromatic-Fielder GEA-Strea-1) for 30 minutes at a temperature of 60° C.

The dried granules were screened in the granulator (Erweka (FGS+AR400E)) over a sieve of 0.315 mm for 5 to 10 minutes at 100 turns per minute.

### Example 2A

### Comparative Example

The granulated product obtained in example 1 was then blended with 1% of magnesium stearate in a Pharmatech equipment at 28 rpm.

The granulated product was tabletted in a tabletting machine (Korsch—PH100) at compression forces varying from 5 kN to 20 kN.

Tablets had a surface of 1 cm², the diameter of the tablet was 11.3 mm and the weight is 350 mg.

### Example 2B

The granulated product obtained in example 1 was then (dry) blended with 2% Ac-di-sol (disintegrant) and 1% of magnesium stearate in a Pharmatech equipment at 28 rpm

The granulated product was tabletted in a tabletting machine (Korsch—PH100) at compression forces varying from 5 kN to 20 kN.

Tablets had a surface of 1 cm², the diameter of the tablet was 11.3 mm and the weight is 350 mg.

### Example 3

### Comparative Example—According to WO 2010/025796

Coarse erythritol product (Cargill C*PharmEridex 16956) was milled in a Bauermeister turbo mill UTL at a 1 mm sieve and powder with a volume mean diameter of 30 μm was obtained.

The volume mean diameter was determined with laser diffraction. The erythritol had a specific surface area of 0.40 m²/g.

500 g of the milled erythritol powder was dry blended in a high Shear Mixer (Pro-C-ept-Mi-Pro, Chopper: 3000 rpm and Impeller: 1200 rpm) for 60 seconds.

79.17 g of liquid sorbitol (at 70% dry substance) (Cargill C*PharmSorbidex NC 16205) was added in droplets at 9.5 g/min). After the addition of the liquid sorbitol, the mixing of the blend was continued for 60 seconds.

The granulated powder was manually wet screened over a 2 mm sieve.

The wet sieved granules were dried in the fluid bed (Aeromatic-Fielder GEA-Strea-1) for 30 minutes at a temperature of 70° C.

The dried granules were screened in the granulator (Erweka (FGS+AR400E) over a sieve of 0.500 mm for 5 to 10 minutes at 100 turns per minute The dry sieved granules were then blended with 2% Ac-di-sol (disintegrant) and 3% of magnesium stearate in a Pharmatech equipment at 28 rpm.

The thus obtained tablets and granulated product from example 2A and 2B and example 3 were analyzed as follows:

Disintegration Time

| Compression | Product from | Product from | |
|---|---|---|---|
| Force | example 2A | example 2B | |
| (kN) | (seconds) | (seconds) | Product from 3 |
| 15 | 127 | 71 | 188 |
| 20 | 143 | 95 | 182 |

By adding the disintegrant, the disintegration time is less than 100 seconds, for tablets of the current invention and prepared at compression force of 20 kN.

The tablets, prepared according to example 3 (comparative example), including erythritol and sorbitol and prepared according to WO 2010/025796, do not have a disintegration time lower than 100 seconds and are thus not suitable for orodispersibility purposes.

Tensile strength

| Compression Force | Product from example | Product from example |
|---|---|---|
| (kN) | 2A (N/mm²) | 2B (N/mm²) |
| 15 | 2.74 | 2.20 |
| 20 | 3.03 | 2.99 |

The tablets from the current invention, including a disintegrant have a lower tensile strength than the tablets without disintegrant. A lower tensile strength corresponds to a lower disintegration time. As a tablet is less compacted, it is easier for the fluid to get into the tablet an dinduce disintegration of the tablet.

1.-11. (canceled)

12. An orodispersible tablet comprising:
a disintegrant;
erythritol; and
at least 10% w/w isomalt,
wherein the orodispersible tablet, when prepared with a compression force of 20 kN and having a surface of one square centimeter and a weight of 350 mg, has a disintegration time of less than 100 seconds, the disintegration time determined according to the European Pharmacopoeia VI, Test method 2.9.1 using a pharmaceutical disintegration tester model ZT 73.

13. The tablet of claim 12, wherein isomalt is present in an amount of less than 50% w/w.

14. The tablet of claim 12, wherein the disintegrant is present in an amount of 0.5 to 20% w/w.

15. The tablet of claim 12, wherein the tablet has at 15 kN a tensile strength of at least 2.5 N/mm², wherein the tensile strength (Ts), expressed as N/mm² is calculated as follows:
Ts=2H/πTD,
wherein H is the hardness, T is the thickness, and D is the diameter of the tablet, and wherein the hardness is determined according to the European Pharmacopoeia VI Test method 2.9.8 using a pharmaceutical hardness tester model Multicheck V.

16. A method for preparing the orodispersible tablet of claim 12, the method comprising:
granulating erythritol, isomalt, and optionally the disintegrant to prepare a granulate;
optionally adding disintegrant to the granulate; and
tabletting the granulate.

17. The method of claim 16, wherein an active ingredient is added prior to and/or after the granulating.

18. A granulate comprising:
disintegrant;
erythritol; and
from 10% w/w to 50% w/w isomalt.

19. The granulate of claim 18, wherein the disintegrant is present in an amount of 0.5 to 5% w/w.

20. The granulate of claim 18, wherein the disintegrant is present in an amount of 1 to 2% w/w.

21. The orodispersible tablet of claim 12, comprising at least 15% w/w isomalt.

22. The orodispersible tablet of claim 12, comprising at least 20% w/w isomalt.

23. The orodispersible tablet of claim 12, wherein the disintegration time is less than 90 seconds.

24. The orodispersible tablet of claim 12, wherein the disintegration time is less than 80 seconds.

25. The orodispersible tablet of claim 12, wherein the disintegration time is less than 60 seconds.

26. The tablet according to claim 12, wherein the disintegrant is present in an amount of 1 to 15% w/w.

27. The tablet of claim 12, wherein the disintegrant is present in an amount of 2 to 10% w/w.

28. The tablet of claim 12, wherein the tablet has at 15 kN a tensile strength of at least 2.7 N/mm².
